(19) United States
(12) Patent Application Publication
(10) Pub. No.: US 2009/0163427 A1
(43) Pub. Date: Jun. 25, 2009

(54) Pharmaceutical Composition of Enterosorbent and Prebiotics, Dosage Forms, and the Method for Prevention and Treatment of Gastrointestinal Disorders

(76) Inventor: Aleksander Vladimirovich Dikovskiy, Moscow (RU)

Correspondence Address: THE NATH LAW GROUP, 112 South West Street, Alexandria, VA, 22314, US

(21) Appl. No.: 12/226,140

(22) PCT Filed: Apr. 10, 2006
(86) PCT No.: PCT/RU2006/000175

§ 371 Date: Jan. 8, 2009

(51) Int. Cl. A61K 45/00; A61P 1/14

(52) U.S. Cl. 514/22

(57) ABSTRACT

The pharmaceutical composition is a combination of hydrolytic lignin with moisture of 55% to 65% consisting of the particles measuring 0.15 mm to 0.55 mm, a 45% to 55% aqueous lactulose solution, and a 50% to 55% aqueous oligosaccharide solution at the following ingredient ratio (weight percent): an aqueous lactulose solution: 10÷60; oligosaccharides: 10÷50; hydrolytic lignin: quantity sufficient. Hydrolytic lignin, lactulose and fructose oligosaccharides are sequentially added and mixed using a rotor blender. The composition is administered orally for no less than 14 days and no more than 30 days, two to four times a day, depending on the patient's weight and age. The composition is used as a medicine for treatment of the gastrointestinal disorders, including bacterial, viral, protozoal enteric infections, food poisoning, acute and chronic hepatitis and cirrhosis, diarrhea, peptic ulcer, Crohn's disease, ulcerative colitis, irritable bowel syndrome, mineral disorders with Ca/Mg deficiency, including osteoporosis and other alterations of the bone formation, as an immunomodulator in atopic dermatitis and immunodeficiency conditions, for protection and recovery of intestinal flora after antibiotic therapy, chemotherapy, and radiotherapy. The result is accelerated achievement of the effect and the enhanced action on the state of intestinal microbiocenosis as well as increased effectiveness of treatment of hepatitis and liver cirrhosis, elimination of undesired adverse effects in clinical usage, and extension of indications, i.e. the extended spectrum of usage in prevention and treatment.

The claimed group of inventions pertains to the field of medicine, veterinary and medical industry and can be applied in prevention and treatment of the gastrointestinal disorders using biologically active preparations.

### PRIOR ART

It is known a composition for preparation of the ready-to-use enterosorbent dosage form containing enterosorbent (activated charcoal) and the sugar (sucrose) aqueous solution (RF Medicinal drug register., Moscow, Infarmkhim, 1993, item 856, column 1).

However, the ready-to-use dosage form obtained out of this known composition easily undergoes bacterial contamination due to the presence of sucrose, a source of carbon, which is utilized by microorganisms as a substrate.

It is known an efficacious enterosorbent, hydrolytic lignin (RF Patent No. 2125463, cl. A 61 K 35178, 1998).

Hydrolytic lignin is a mixture of substances that includes lignin of a plant cell, a portion of polysaccharides, a group of substances of lignohumic complex, simple sugars unwashed after hydrolysis, mineral and organic acids, ashes, and other substances. Hydrolytic lignin is characterized by a large pore diameter very similar to wood charcoal porosity and high reactivity and intended for treatment of acute poisoning. In some cases, administration of hydrolytic lignin is accompanied by undesired adverse events involving by-effects in the gastrointestinal tract. For this reason, the medical usage of lignin is limited.

It is known the method for preparation of the ready-to-use dosage form of the enterosorbent, hydrolytic lignin, including drying-up and packing with regard to the ready-to-use dosage form of the enterosorbent. This known method excludes introduction of any additional ingredients into the composition.

It is known lactulose, a synthetic disaccharide the molecule of which consists of galactose and fructose (Reference book Medicinal drugs and their usage [composition, properties, administration, interaction, contraindications], Moscow, “Sezam-marketing” Publishing house, 1998, item 423).

Lactulose is a disaccharide of lactose consisting of galactose and fructose. Lactulose has a chemical formula of C₁₂H₂₂O₁₁ and molecular weight of 342.3. The lactulose molecule consists of galactose and fructose residues linked by a glycoside bond. According to modern classification of carbohydrates, its chemical name is 4-O-beta-D-galactose pyranosyl-D-fructose. This sugar doesn't exist in nature and is a disaccharide synthesized out of lactose. In food-processing industry, lactulose is used as a biologically active additive to children's formulas and dietetic diary products. However, dietary products are not used for correction of the state of intestinal microbiocenosis.

There are known oligosaccharides, mostly short-chain fructose oligosaccharides (FOS) consisting of one to three fructose molecules and linked to one sucrose molecule. Their polymerization degree is less than five carbohydrate residues in a chain. FOS are natural components of many vegetables and fruits (onion, artichoke, garlic, bananas, etc); they are used in dietary products and children's products as a factor stimulating growth of normal intestinal flora (Mitsuoka et al, 1987; Gibson et al, 1995).

It is known the method of treatment for disorders of the digestive system, including administration of hydrolytic lignin (RF patent No. 2026078, cl. A 61, K 35/78, 1995).

However, it is known from clinical practice that hydrolytic lignin administration can cause constipation, mucosal irritation, and other undesired effects.

Also, it is known the pharmaceutical composition, the method for preparation of the dosage form, and the method for prevention and treatment of the disorders of the digestive system. The pharmaceutical composition contains hydrolytic lignin, 60 to 90 weight percent, and lactulose, 10% to 40%. The method of preparation includes addition of hydrolytic lignin to lactulose syrup and mixing the mass using a high-speed blender. The method for prevention and treatment of the disorders of the digestive system includes administration of the formulation with indicated composition, obtained by the indicated method, in the amount of 10 g/kg of the laboratory guinea pig for 10 days (RU No. 2131260, 1999—prototype).

Shortcomings of this known group of technical solutions are slow achievements of significant results and the fact that the experimental data are obtained using exclusively a guinea pig model whose intestinal flora considerably differs from human flora. There are limited data characterizing certain formulations and dosage forms of preparations based on enterosorbents and prebiotics. There are no clinical studies that directly confirm the efficacy of the suggested sorbents and prebiotics in dysbioses of the various etiologies in humans.

The technical task of the claimed group of inventions linked by a single inventive conception is a development of the efficient pharmaceutical composition [a drug or biologically active food additive for usage in medicine and veterinary], the method of preparation of ready-to-use dosage forms of combined medicines, the method for prevention and treatment of the disorders associated with abnormal intestinal microbiocenosis as well as extension of indications for enterosorbent and prebiotics administration in human disorders caused by dysbioses.

The technical result ensuring the solution for the task posed consists of accelerated achievement of significant results, increased efficacy of action on the state of intestinal microbiocenosis, increased efficacy of treatment of chronic hepatitis and liver cirrhosis, especially in elder patients, elimination of undesired adverse effects when used in clinical practice, and extension of indication for medical usage, i.e. extension of the spectrum of efficient usage for prevention and treatment of bacterial, viral, and protozoal intestinal infections, food poisoning, acute and chronic hepatitis and liver cirrhosis, diarrhea, peptic ulcer, Crohn's disease, ulcerative colitis, irritable bowel syndrome, impaired mineral disorders associated with Ca/Mg deficiency, including osteoporosis and other disorders of the bone formation, as an immunomodulator in atopic dermatitis and immunodeficiency conditions, for protection and recovery of intestinal flora after antibiotic therapy, chemotherapy, and radiotherapy, for prevention and treatment of porto-systemic encephalopathy.

### SUMMARY OF THE INVENTION

The nature of invention with regard to the pharmaceutical composition is that it contains hydrolytic lignin of 55% to 65% moisture consisting of particles measuring 0.15 to 0.55 mm, a 45% to 55% aqueous lactulose solution, and a 50% to 55% aqueous oligosaccharide solution with the following ingredient ratio (weight percent):
an aqueous lactulose solution—10÷60;
oligosaccharides—10÷50;
hydrolytic lignin—quantity sufficient.

Preferably, the pharmaceutical composition contains short-chain fructose oligosaccharides (FOS), is prepared in a form of wet powder, and contains hydrolytic lignin consisting of particles measuring 0.15 mm to 0.55 mm.

The nature of invention with regard to the preparation method of a pharmaceutical composition containing hydrolytic lignin, lactulose, and fructose oligosaccharides, is that hydrolytic lignin, lactulose, and fructose-oligosaccharides are sequentially added and mixed using a rotor blender up to 100 l in volume at the temperature of 30° C. for 20 minutes at a rotation speed of the rotor blender up to 40 rev/min with subsequent granulation and drying-up at the shelf drier at the temperature of 60° C. until the residual granule moisture of 3.0% is reached. Then, the obtained granules are packed into the bags.

Preferably, lactulose is mixed as a 45% to 55% aqueous solution which is added to hydrolytic lignin in the amount of 10 to 60 weight percent; hydrolytic lignin of the moisture of 55% to 65% consisting of particles measuring 0.15 mm to 0.55 mm is taken, or hydrolytic lignin consisting of particles measuring 0.15 mm to 0.55 mm is taken, then, the dosage form is dried until the moisture of 50% to 70% is reached, then, the obtained mass is packed in a form of powder or tablets; or hydrolytic lignin consisting of particles measuring 0.15 mm to 0.55 mm is taken, then, the dosage form is dried until the moisture of 45% to 60% is reached, the obtained mass is additionally granulated and packed in a form of wet granules; or hydrolytic lignin consisting of particles measuring 0.15 mm to 0.55 mm is taken, the dosage form is obtained in a form of paste and packed into the tubes and glass containers.

The nature of invention with regard to another way of presentment of pharmaceutical composition is that it is made in a form of tablets, including enteric-coated and chewable tablets, powders, granules, paste, suspension, syrup, pills, capsules, and suppositories.

The nature of invention with regard to the method for prevention and treatment of the disorders of the digestive system is that the above mentioned pharmaceutical composition is administered orally for no less than 14 days and no more than 30 days, 2 to 4 times a day, depending on a patients weight and age.

Preferably, the daily dose of hydrolytic lignin, lactulose, and fructose oligosaccharides as ingredients of the composition for prevention or treatment is, respectively:
hydrolytic lignin—1.0 g to 5.0 g
lactulose—0.5 g to 5.0 g
fructose oligosaccharides—0.5 g to 10.0 g.

The nature of invention with regard to the way of administration of above-mentioned pharmaceutical composition is that it is used as a medicinal preparation for treatment of the gastrointestinal disorders, including intestinal bacterial, viral, or protozoal infections, food poisoning, acute and chronic hepatitis and cirrhosis, diarrhea, peptic ulcer, Crohn's disease, ulcerative colitis, irritable bowel syndrome, mineral disorders with Ca/Mg deficiency, including osteoporosis and other types of impaired bone formation; as an immunomodulator, in atopic dermatitis and immunodeficiency conditions; for protection and recovery of intestinal flora after antibiotic therapy, chemotherapy, or radiotherapy.

Preferably, the medicinal preparation containing the pharmaceutical composition per any of items 1-3, 10, prepared per any of items 4-9, is used in combination with other medicinal drugs, antibiotics, vitamins, minerals, amino acids, proteins, fats, carbohydrates and/or food products.

The properties of the claimed compositions, the method for preparation, and the method for prevention and treatment are determined by the used ingredients and their synergic action on the body.

Lignin exerts “nonspecific” sorption, i.e. in addition to binding toxic substances in the intestines; it concurrently eliminates useful biologically active substances, such as enzymes, vitamins, amino acids, etc.

The therapeutic/prophylactic properties of lactulose are determined by the fact that it doesn't undergo processing in the upper gastrointestinal tract (no lysis by intestinal enzymes), but is passed unchanged into the large bowel where it is actively utilized by normal intestinal flora, namely, by lactobacillus spp. and bifidobacterium spp. This (at the indicated lactulose content) prevents depletion of flora due to lignin exposure. In the large bowel, lactulose is utilized by bacteria with formation of low-molecular organic compounds, mostly, organic acids (lactic, acetic, butyric, propionic) that cause the decrease in pH of the intestinal contents. Due to acidification of the intestinal contents, osmotic pressure rises, and intestinal contents liquefy. Organic acids formed by bacteria inhibit growth of proteolytic bacteria, thus, reducing the quantity of toxic products of protein metabolism, including skatole, n-cresol, indole, and phenol.

Since oligosaccharides, particularly short-chain fructose oligosaccharides (consisting out of one to three fructose molecules linked to one sucrose molecule), are larger than disaccharides molecules (lactulose), their presence (at the indicated percent content determining weight ratio to lignin and lactulose) allows to extend the spectrum of action of the composition and increase its substrate value for intestinal flora. An oligosaccharide fermentation process results in more marked stimulating effect of the composition on the state of intestinal microbiocenosis. Thus, within the large bowel, there is an increase in production of acetic, formic, lactic, and fatty acids which, combined with lactulose fermentation products, inhibit growth of the wide spectrum of saprophytic microorganisms and pathogens (Salmonella spp, Shigella spp, fungi). By inhibiting growth of proteolytic bacteria, these products markedly reduce ammonia levels, decreasing the toxin load on the liver and protecting the brain. Oligosaccharide fermentation products additionally stimulate peristalsis, contribute to the chime liquefaction relieving constipation, reduce the load on the kidneys, and stimulate the immune response. The presence of oligosaccharides promotes synthesis and utilization of vitamins A and E by microorganisms constituting normal intestinal flora.

The most effective quantitative ratio of ingredients in the composition, the particle size, lignin moisture, and lactulose/fructose oligosaccharide concentration as well as the regimen of preparation and therapeutic usage of the composition are chosen on the basis of laboratory and clinical studies. In particular, for the first time, clinical studies have shown an additional effect of enterosorbent and prebiotics.

### DETAILED DESCRIPTION OF THE INVENTION

Below, there are examples of the invention implementation.

### EXAMPLE 1

The ready-to-use dosage form was prepared in a form of the wet powder. The composition contained 80 weight percent of hydrolytic lignin of the moisture of 55%, containing particles measuring 0.15 mm to 0.55 mm and 20 weight percent of 45% aqueous lactulose solution, and short-chain fructose oligosaccharides. After mixing in the rotor blender, the obtained mixture was dried up until the 50% moisture was reached and packed into the bags made of laminated foil. The ready-made bags underwent sterilization.

### EXAMPLE 2

Following the technique described in Example 1, the dosage form was prepared in a form of the wet hydrolytic lignin powder with the 65% moisture consisting of the particles measuring 0.15 mm to 0.55 mm and taken in an amount of 90 weight percent. To hydrolytic lignin, 10 weight percent of 55% aqueous lactulose and short-chain fructose oligosaccharides were added. The mixture was dried up until the 70% moisture was reached and packed into the bags made of laminated foil and, then, sterilized.

### EXAMPLE 3

Following the technique described in Example 1, the pharmaceutical composition was prepared, dried up until the 45% moisture was reached, and passed through the granulating machine. The obtained granulation wet mass was packed into the bags made of laminated foil and sterilized.

### EXAMPLE 4

Following the technique described in Example 2, the composition was prepared, after drying up until the 60% moisture was reached, passed through the granulating machine, packed into the bags made of laminated foil and sterilized.

### EXAMPLE 5

The mixture of hydrolytic lignin containing particles measuring 0.15 mm to 0.55 mm and an aqueous lactulose solution, short-chain fructose oligosaccharides, was prepared. After careful mixing in a rotor blender, the obtained mixture with 75% moisture appeared as a paste that was packed into tubes and sterilized.

### EXAMPLE 6

At the Moscow Scientific Research Institute of Pediatrics and Pediatric Surgery, the clinical trial of the developed dosage forms of lignin containing lactulose, Examples 1 to 5 was performed. The trial was conducted on children at the age of 3 to 15 years old who had alterations of intestinal flora. The children were under supervision of gastroenterologists. All children had been repeatedly admitted to the hospital or received outpatient treatment for the diagnosis of “a gastrointestinal disorder”. The symptoms were intermittent abdominal pain, diarrhea, and flatulence. Microbiology revealed a low content of bifidobacteria in the large bowel (no more than 10² CFU/g). Also, examination included clinical assessment and instrumental investigations (endoscopy, abdominal ultrasound scanning, stool examination for bacteriology).

The group of 60 children was divided into an experimental and control groups, including 45 and 15 participants, respectively. Children from an experimental group received the tablets of combined preparation (3.8 g to 4.2 g of hydrolytic lignin+0.73 to 0.77 g of lactulose per one dose) for two weeks. The control group received standard treatment for this disorder (antispasmodics and bacterial preparations).

After completion of the treatment course with the enterosorbent and prebiotic, the children were assessed using clinical and microbiological parameters (bifidobacteria and lactobacillus titers in stool).

Microbiological examination revealed an increase in bifidobacteria and lactobacillus titers from 10² CFU/g to 10⁶ CFU/g in children in the experimental group at the end of treatment. In these children, diarrhea, flatulence, and abdominal pain disappeared. Good and satisfactory clinical results were seen in 84% and 16% of cases, respectively.

The condition of children from the control group somewhat improved, but microbiological parameters reflecting the state of intestinal flora changed insignificantly. Intermittently, the children experienced intestinal dysfunction. Out of ethical issues, the control group of children was given lignin with lactulose in tested doses after completion of the clinical trial. As a result, their microbiological parameters gradually improved and approached the ones observed in the experimental group.

### EXAMPLE 7

At the department of outpatient pediatrics of the Saratov State Medical University, clinical evaluation of the efficacy of lignin-lactulose combination in combined therapy of atopic dermatitis in children was performed.

The study included 40 children at the age of 6 months to 3 years old with a diagnosis of atopic dermatitis.

Severity of the disorder was assessed using a SCORAD scale. In all patients, examination revealed polyvalent allergy to domestic and plant antigens. The children at the age of 1 to 3 years old received oral suspension containing lactulose, 60 mg, and lignin, 450 mg, three times a day; the children after 3 years of age were given suspension containing lactulose, 120 mg, and lignin, 900 mg. After treatment with the enterosorbent and lactulose, condition of the children significantly improved. In fact, the children with mild and moderate atopic dermatitis showed improvement of major symptoms (pruritis, erythema, edema, etc) in 84% and 76%, respectively. On the fourteenth day, persistence of mild erythema was noted only in three cases.

### EXAMPLE 8

Clinical evaluation of the enterosorbent efficacy with prebiotic was performed for patients with chronic hepatitis and the liver cirrhosis at the department of field therapy of the State Institute of post-graduate education for physicians, RF Ministry of Defense (Moscow). Twenty five patients at the age of 18 to 60 years old were under supervision for confirmed diagnosis of chronic hepatitis/liver cirrhosis. All the patients had chronic alcoholism in the history. Fifteen patients with hepatitis and liver cirrhosis in the experimental group received combined preparation of lignin, 2.6 g, lactulose, 0.4 g, and fructose oligosaccharides, 1.8 g, 4 times a day for one month in addition to the basic therapy (Carsil, Essentiale, vitamins of B group).

Ten patients in the control group received only basic therapy. The clinical study showed that the enterosorbent with lactulose and fructose oligosaccharides alleviated symptoms and signs of hepatic encephalopathy in 44% of patients, contributed to improved intestinal flora in 68% as well as improved the function of the gastrointestinal tract and was well-tolerated by all the patients.

Implementation of this group of inventions results in accelerated achievement of significant results and increased efficacy of influencing intestinal microbiocenosis as well as more effective treatment of chronic hepatitis and cirrhosis, particularly in elder patients, elimination of undesired adverse effects when used in clinical practice, and extension of indications, i.e. the extended spectrum of effective usage in prevention and treatment of bacterial, viral, protozoal enteric infections, food poisoning, acute and chronic hepatitis and cirrhosis, diarrhea, peptic ulcer, Crohn's disease, ulcerative colitis, irritable bowel syndrome, mineral disorders with Ca/Mg deficiency, including osteoporosis and other alterations of the bone formation, as an immunomodulator in atopic dermatitis and immunodeficiency conditions, for protection and recovery of intestinal flora after antibiotic therapy, chemotherapy, and radiotherapy, for prevention and treatment of porto-systemic encephalopathy.

Thus, the invention allowed to obtain the following product: Pharmaceutical composition of enterosorbent based on hydrolytic lignin and combined with lactulose and fructose oligosaccharides as well as the method of effective treatment of the disorders associated with impaired microbiocenosis in the gastrointestinal tract.

### INDUSTRIAL APPLICATIONS

This invention is embodied with the use of multifunctional and easily available up-to-date equipment and substances that are widely used in the industry and health care.

1.-14. (canceled)

15. A pharmaceutical composition for the prophylaxis and/or treatment of gastrointestinal tract disease caused by a disturbance of intestinal microbiocenosis; a disturbance of mineral metabolism with the deficiency of calcium and magnesium; or atopic dermatitis, the pharmaceutical composition comprising:
lactulose in an amount of 10-60% by weight;
fructooligosaccharide in an amount of 10-50% by weight; and
hydrolyzed lignin.

16. The pharmaceutical composition of claim 15, wherein the disturbance of intestinal microbiocenosis is selected from the group consisting of acute and chronic hepatitis, cirrhosis, enteric infection of bacterial, viral and protozoal etiology, stomach ulcer, duodenal ulcer, Crohn's disease, nonspecific ulcerative colitis, and irritable colon syndrome.

17. A method for the prophylaxis and/or treatment of a gastrointestinal tract disease in a patient caused by a disturbance of intestinal microbiocenosis selected from the group consisting of acute and chronic hepatitis, cirrhosis, enteric infection of bacterial, viral and protozoal etiology, stomach ulcer, duodenal ulcer, Crohn's disease, nonspecific ulcerative colitis, and irritable colon syndrome; a disturbance of mineral metabolism with the deficiency of calcium and magnesium; intestinal dysbiosis; or atopic dermatitis; the method comprising:
orally administering a therapeutically effective amount of the pharmaceutical composition of claim 15 to a patient in need thereof 2-4 times a day for a period of 14-30 days.

18. The method of claim 17, wherein the gastrointestinal tract disease is caused by intestinal dysbiosis after one or more of antibiotic therapy, radiotherapy and chemotherapy.

19. The method of claim 17, further comprising:
administering to the patient one or more members selected from the group consisting of an antibiotic, a vitamin, a microelement, an amino acid, a protein, a fat, a carbohydrate and a food product.

20. A method for preparing the pharmaceutical composition of claim 15, comprising:
successively adding lactulose to hydrolyzed lignin and fructooligosaccharide to form an admixture; and
mixing the admixture using a rotary blender at a temperature of 30° C. for 20 minutes at 40 rotations per minute, to form a blended mixture.

21. The method of claim 20, wherein the lactulose is a 45-55% aqueous solution.

22. The method of claim 20, wherein the hydrolyzed lignin has a moisture content of 55-65% and a particle size of from 0.15 to 0.55 mm.

23. The method of claim 20, wherein the fructooligosaccharide is a 50-55% aqueous solution.

24. The method of claim 20, wherein the pharmaceutical composition is in the form of granules, the method further comprising:
granulating the blended mixture to form granules;
drying the granules at a temperature 60° C. to produce granules having 3% residual moisture; and
packaging the obtained granules.

25. The method of claim 20, wherein the pharmaceutical composition is in the form of a powder, the method further comprising:
drying the mixture to a moisture content of 50-70% to obtain a powder; and
packaging the obtained powder.

26. The method of claim 20, wherein the pharmaceutical composition is in the form of wet granules, the method further comprising:
drying the mixture to a moisture content of 45-60%; and
granulating the mixture to obtain wet granules.

27. The method of claim 20, wherein the pharmaceutical composition is in the form of a wet paste, the method further comprising:
packaging the blended mixture in the form of a wet paste into tubes or into glass containers.

28. The method of claim 17, wherein the gastrointestinal tract disease is caused by osteoporosis.
